(19) United States
(12) Patent Application Publication
(10) Pub. No.: US 2014/0255246 A1
(43) Pub. Date: Sep. 11, 2014

(54) MEDICAL DEVICE HAVING NIOBIUM NITINOL ALLOY

(71) Applicant: ABBOTT LABORATORIES, Abbott Park, IL (US)

(72) Inventors: John A. Simpson, Carlsbad, CA (US); John F. Boylan, Murrieta, CA (US)

(73) Assignee: ABBOTT LABORATORIES, Abbott Park, IL (US)

(21) Appl. No.: 13/791,860

(22) Filed: Mar. 8, 2013

(51) Int. Cl. A61M 25/09; C22C 30/00; C22F 1/10; C22C 19/03

(52) U.S. Cl. CPC A61M 25/09; C22C 19/03; C22C 30/00; C22F 1/10
USPC 420/441; 420/580; 148/676; 148/707; 148/426; 148/442; 72/362

(57) ABSTRACT

Guide wire devices and other intra-corporal medical devices fabricated from a Ni—Ti—Nb alloy and methods for their manufacture. The Ni—Ti alloy includes nickel, titanium, and niobium either up to its solubility limit in Ni—Ti, or in amounts over 15 atomic percent so as to provide a dual phase alloy. In either case, the Ni—Ti—Nb alloy provides increased stiffness to provide better torque response, steerability, stent scaffolding strength, and similar properties associated with increased stiffness, while still providing super-elastic or linear pseudo-elastic properties.

### BACKGROUND

Guide wires are used to guide a catheter for treatment of intravascular sites such as PTCA (Percutaneous Transluminal Coronary Angioplasty), or in examination such as cardio-angiography. For example, a guide wire used in the PTCA is inserted into the vicinity of a target angiostenosis portion together with a balloon catheter, and is operated to guide the distal end portion of the balloon catheter to the target angiostenosis portion.

A guide wire needs appropriate flexibility, pushability and torque transmission performance for transmitting an operational force from the proximal end portion to the distal end, and kink resistance (resistance against sharp bending). To meet such requirements, superelastic materials such as a nitinol alloy and high strength materials have been used for forming a core member (wire body) of a guide wire.

Some near equi-atomic binary nickel-titanium alloys are known to exhibit “super-elastic” (sometimes referred to as “non-linear pseudo-elasticity) behavior by virtue of a reversible, isothermal stress-induced austenitic to martensitic transformation.

Super-elastic nitinol alloys may exhibit upwards of 8% elastic strain (fully-recoverable deformation). At room or body temperature and under minimal stress the material assumes a crystalline microstructure structure known as austenite. As the material is stressed, it remains in the austenitic state until it reaches a threshold of applied stress (a.k.a. the “upper plateau stress”), beyond which the material begins to transform into a different crystal structure known as martensite. Upon removal of the applied stress, the martensite reverts back to the original austenite structure with an accompanying return to essentially zero strain (i.e., the original shape is restored).

While nitinol exhibits very high elastic strain limits so as to be quite resistant to kinking, the material exhibits an elastic modulus that is lower than for stainless steel, making nitinol generally less effective at transmitting torque to the guide wire tip. For example, a nitinol guide wire has a greater tendency than stainless steel to elastically absorb a significant amount of applied torque or twist as opposed to directly transmitting torque from one end to the other. Further, nitinol has only moderate plateau stress levels, and is therefore less resistant to bending forces (as compared to stainless steel), and thus is less effective at providing support as a guide wire for catheter delivery or as a stent for arterial scaffolding.

### BRIEF SUMMARY

The present disclosure describes intra-corporal medical devices (e.g., guide wires, stents, embolic protection filters, graft assemblies, etc.) and methods for their manufacture. Such devices may include a body, at least a portion of which is fabricated from a Ni—Ti alloy comprising nickel, titanium, and niobium. In an embodiment, the niobium may be present in the Ni—Ti alloy in an amount not more than a solubility limit of the Nb in Ni—Ti so that the alloy comprises only a single phase (e.g., an ordered nickel-titanium phase with a relatively small atomic fraction of niobium present therein). The solubility limit of Nb in Ni—Ti is believed to be from about 3 atomic percent to 3.5 atomic percent.

Because of the presence of niobium, the Ni—Ti—Nb alloy has elastic moduli values (e.g., Young's modulus and shear modulus) that are considerably higher than comparable binary Ni—Ti alloy under otherwise similar conditions (e.g., same level of cold work, etc.). Elastic moduli values may be increased as compared to the comparable binary Ni—Ti alloy for both austenitic and martensitic states. The resulting alloy is highly durable, corrosion resistant, with greater stiffness levels as compared to comparable binary Ni—Ti. In the context of guide wires, these characteristics facilitate guiding the guide wire through tortuous anatomy. In the context of a stent, these characteristics provide increased stiffness so as to better provide scaffolding support to a body lumen. Such characteristics are similarly advantageous in other intra-corporal medical devices, such as embolic protection filters, graft assemblies, etc.

According to another embodiment, at least a portion of the body of the intra-corporal medical device may be fabricated from a Ni—Ti alloy comprising nickel, titanium, and niobium, and in which the niobium is present in an amount of at least 15-atomic percent. In such embodiments, the Ni—Ti—Nb alloy comprises two phases, because the niobium is present at levels above the solubility limit of niobium in Ni—Ti. A first (e.g., primary) phase is Ni—Ti rich (e.g., including only a small fraction of niobium, at the Nb solubility limit). A second phase that is more Nb rich is also present (although Ni—Ti is still the predominant phase). While the Ni—Ti rich first phase may exhibit super-elastic properties (e.g., where transformation between austenitic/martensitic states is possible), the Nb rich second phase does not exhibit super-elastic properties, but conventional mechanical elasticity property characteristics.

According to the rule of mixtures, the resulting two phase material exhibits elastic moduli values that are considerably higher than comparable binary Ni—Ti alloy, which stiffness depends on the relative volume fraction of the two phases. As a result, the two-phase Ni—Ti—Nb alloy has higher stiffness and better torque response and steerability (important in guide wires) as compared to binary Ni—Ti alloys. Preferably, the niobium content in “high” niobium alloys is not more than 35 atomic percent, not more than 30 atomic percent, or not more than the niobium content of the Ni—Ti—Nb quasi-binary eutectic composition (e.g., believed to be about 26 atomic percent Nb). At such niobium concentrations, the super-elastic Ni—Ti still makes up the vast majority of the Ni—Ti—Nb alloy, whereby super-elastic characteristics of the overall Ni—Ti—Nb alloy can be maintained.

In an embodiment, the two-phase Ni—Ti—Nb alloy may be cold worked to inhibit further stress induced martensitic phase transformation, so that the bulk alloy may exhibit linear pseudo-elastic properties. Of course, alternatively, cold work may be limited (or subsequent heat treatments provided) to retain super-elastic characteristics provided by the Ni—Ti rich phase. While the second phase (i.e., that is richer in Nb than the Ni—Ti phase including Nb at its solubility limit—e.g., 3 to 3.5 atomic percent) may not be super-elastic, it is relatively stiff, ductile, and strong, exhibiting conventional elasticity characteristics, and provides an increase in stiffness properties to the two-phase alloy which it is included in. Advantageously, the two phases are coherent with one another, being metallurgically bonded to one another.

In another embodiment, a method for fabricating an intra-corporal medical device is disclosed. The method includes fabricating a medical device body where at least a portion of the body comprises a nickel-titanium alloy comprising nickel, titanium, and niobium. The nickel-titanium-niobium alloy may comprise a “low” niobium alloy (i.e., niobium present at no more than its solubility in Ni—Ti) or “high” niobium alloy (i.e., niobium present at least 15 atomic percent so that the Ni—Ti—Nb alloy includes a Ni—Ti rich first phase and a second phase in which Nb content is relatively richer) as described above.

In a particular method, the second phase (with higher Nb content than the nearly pure Ni—Ti first phase) consists of fine particles which serve to reinforce a matrix that is comprised of the Ni—Ti rich first phase, which exhibits super-elastic properties. The resulting microstructure allows the bulk Ni—Ti—Nb alloy to exhibit super-elastic or linear pseudo-elastic behavior, while providing increased stiffness according to the rule of mixtures as a result of inclusion of the relatively stiff and ductile second phase.

These and other objects and features of the present disclosure will become more fully apparent from the following description and appended claims, or may be learned by the practice of the embodiments of the invention as set forth hereinafter.

### BRIEF DESCRIPTION OF THE DRAWINGS

To further clarify the above and other advantages and features of the present disclosure, a more particular description of the invention will be rendered by reference to specific embodiments thereof which are illustrated in the appended drawings. It is appreciated that these drawings depict only illustrated embodiments of the invention and are therefore not to be considered limiting of its scope. Embodiments of the invention will be described and explained with additional specificity and detail through the use of the accompanying drawings in which:

FIG. 1 illustrates a partial cut-away view of a guide wire device according to one embodiment of the present disclosure;

FIGS. 2A-2C show stress-strain curves for various materials;

FIG. 3 is a side elevation view, in partial cross-section, of a delivery catheter within a body lumen having a stent disposed about the delivery catheter according to an embodiment of the present disclosure;

FIG. 4 is an elevational view, partially in section, of an expanded stent, wherein the stent is implanted within a body lumen after withdrawal of a delivery catheter;

FIG. 5 is a side view of a stent, wherein the stent is in an unexpanded state;

FIG. 6 is a side view of the stent of FIG. 5 in an expanded condition, depicting cylindrical rings connected by undulating links;

FIG. 7 is a side view of a stent depicting cylindrical rings at the end of the stent having a thicker cross-section than the rings at the center of the stent;

FIG. 8 is a plan view of a flattened stent, illustrating a combination of undulating links and straight links;

FIG. 9 is a perspective view of a stent, depicting cylindrical rings connected by straight links;

FIG. 10 depicts a longitudinal plan view of an embodiment of an expanded embolic protection device, including expandable struts;

FIG. 11 depicts a longitudinal plan view of the embolic protection device of FIG. 10, wherein the device is collapsed for delivery into a corporal lumen;

FIG. 12 depicts a perspective view of a graft assembly, including a plurality of attachment systems;

FIG. 13A shows an exemplary as cast Ni—Ti—Nb alloy micro-structure in which primary dendrites of a first NiTi rich phase plus a eutectic mixture of the first phase and a second Nb rich phase are present; and

FIG. 13B shows how the Ni—Ti—Nb alloy forms an elongated microstructure comprised of the Ni—Ti rich primary phase and the eutectic mixture of both phases upon rolling (e.g., hot rolling) of the dual phase microstructure of FIG. 13A.

### DETAILED DESCRIPTION

### I. Introduction

In one aspect, the present disclosure describes intra-corporal medical devices and methods for their manufacture. Intra-corporal medical devices include a body, at least a portion of which is fabricated from a Ni—Ti alloy that includes nickel, titanium, and niobium, where niobium is present in the Ni—Ti alloy in an amount not more than a solubility limit of the Nb in Ni—Ti so that the alloy comprises only a single phase.

According to another embodiment, niobium may be included within the Ni—Ti alloy at amounts well above the solubility limit, for example at or near a concentration of a Ni—Ti—Nb quasi-binary eutectic composition. For example, Nb may be present in an amount of at least 15 atomic percent, about 26 atomic percent, or even in amounts above 26 atomic percent. It may be desirable to limit formation of primary dendrites of the Nb-rich second phase that may occur where niobium is present in amounts over the Ni—Ti—Nb quasi-binary eutectic composition, believed to be about 26 atomic percent niobium. For example, the quasi-binary eutectic composition may be 38Ni36Ti26Nb at 1170° C. (see Powder Metallurgy and Metal Ceramics, 34 (1995), p. 155). As such, in an embodiment, the niobium content may be not more than 35 atomic percent, not more than 30 atomic percent, or not more than the niobium content of the quasi-binary Ni—Ti—Nb eutectic composition (e.g., believed to be about 26 atomic percent). By limiting the niobium content to no more than the concentration of niobium in the Ni—Ti—Nb quasi-binary eutectic, the formation of relatively coarse regions of the Nb-rich phase is prevented, which might otherwise prevent the bulk alloy composition from exhibiting homogeneous super-elastic characteristics with substantially complete strain recovery, which is desirable.

In an embodiment, Nb may be present in an amount somewhat nearer the quasi-binary eutectic composition (e.g., at least 16 atomic percent, at least 18 atomic percent, or at least 20 atomic percent), providing further increased stiffness than at 15 atomic percent.

### II. Low Nb Single Phase Ni—Ti—Nb Alloys

As described above, in an embodiment, the Nb content is limited to no more than its solubility limit in Ni—Ti. Because the Nb is present at a level no greater than the Nb solutility limit in Ni—Ti, no Nb-rich second phase is formed. This advantageously prevents formation of a non-superelastic second phase, which can raise issues to be contended with. As such, in an embodiment, only a single phase Ni—Ti—Nb alloy is formed by limiting the Nb content. For example, the presence of a second phase that cannot be super elastic, in some circumstances, may reduce the ability of such an alloy to completely recover imparted strain and thus to return exactly to its original shape. For example, the second phase particles may become permanently deformed under applied stress, and thereby impede the martensite to austenite reverse transformation upon removal of an applied stress. This may potentially cause incomplete strain recovery (a.k.a. permanent set), which is particularly undesirable in guide wires and some other intra-corporal medical device applications. Furthermore, unless particular care is taken, a two-phase alloy may exhibit less corrosion resistance as compared to a single phase alloy due to localized galvanic effects. Finally, a two-phase alloy may tend to exhibit non-uniform material removal rates during chemical or electrochemical treatments such as electropolishing or etching, likely resulting in undesirable final surface conditions for devices where a smooth finish is desirable, such as stents and embolic protection devices. Such effects need not be of concern for Ni—Ti—Nb alloys in which the Nb is present at a level no greater than its solubility limit in Ni—Ti.

### III. High Nb Dual Phase Ni—Ti—Nb Alloys

As described above, according to another embodiment, where particular care is taken, niobium may be included within the Ni—Ti alloy at amounts well above the solubility limit, for example at or near a concentration of a Ni—Ti—Nb quasi-binary eutectic composition. For example, Nb may be present in an amount of at least 15 atomic percent, from 15 atomic percent to about 35 atomic percent, from 15 atomic percent to about 30 atomic percent, or from 15 atomic percent to about 26 atomic percent. In an embodiment, niobium content is not greater than 35 atomic percent, not greater than 30 atomic percent, or not greater than the niobium content in the Ni—Ti—Nb quasi-binary eutectic (e.g., believed to be about 26 atomic percent niobium).

Where the Nb is present in an amount of at least 15 atomic percent, the Ni—Ti—Nb alloy comprises a Ni—Ti rich first phase that includes Nb at its solublity limit (e.g., believed to be from 3 atomic percent to 3.5 atomic percent) and a second phase that is more Nb rich than the first phase. Because the overall Nb content may be limited to no more than about 35 atomic percent, or about 30 atomic percent, or about 26 atomic percent, the second phase may still be dominated by Ni—Ti. The second phase may not exhibit super-elastic properties, but conventional elasticity characteristics. Because two-phases are present, careful control and care during manufacture of guide wires and similar intra-corporal medical devices fabricated from such two phase alloys may be needed to minimize difficulties associated with corrosion resistance and risk of unwanted permanent set.

Where such appropriate care is taken, advantageously, the dual phase microstructure with Ni—Ti rich and a relatively Nb richer phase yields a bulk alloy material having elastic moduli values that are considerably higher than comparable binary Ni—Ti alloy, according to the rule of mixtures. A comparable binary Ni—Ti-alloy may be one in which the Ni to Ti ratio is equal to that of the Ni—Ti—Nb alloy. For comparison, such dual phase microstructure bulk alloys also exhibit considerably higher elastic moduli values as compared to the above described low Nb Ni—Ti—Nb alloys which include Nb at a concentration up to its solubility limit in Ni—Ti. As a result, the “high” Nb Ni—Ti—Nb alloy may have even better torque response and steerability for guide wires formed therefrom, and better scaffolding strength for stents formed therefrom.

According to one embodiment, in order to minimize the above described concern relative to “permanent set”, the two phases may be specifically arranged within the device body in a thinly layered lamellar structure where relatively thin layers of one phase are surrounded by layers of the other phase. It is believed that even though the Nb richer second phase exhibits only conventional elasticity characteristics, its relatively thin layered, lamellar micro-structure, in which it is surrounded by the Ni—Ti rich phase (which can retain super-elastic or linear pseudo-elastic characteristics depending on degree of cold working) results in an overall bulk alloy structure that is capable of responding as desired under use conditions. In other words, the bulk alloy can exhibit super-elastic or linear pseudo-elastic characteristics. The overall bulk alloy structure provides increased elastic moduli values according to the rule of mixtures based on the elastic moduli values of the two phases and their volume fractions within the bulk alloy. In addition, the plateau stress level associated with such a super-elastic dual-phase alloy may be increased as compared to the binary Ni—Ti material. As a result, guide wires formed therefrom provide better steerability and better torque response when being maneuvered into position, along with improved support when subsequently delivering catheters, while still providing excellent kink resistance as compared to the comparable binary Ni—Ti material.

According to the rule of mixtures, by blending the relatively low modulus (but super-elastic) Ni—Ti matrix with relatively high modulus regions or layers including Nb-rich second phase, the resulting composite structure has elastic moduli characteristics in between the two, where the property change is proportional to the volume fraction of each phase.

In an embodiment, the Ni to Ti ratio is maintained at approximately equal atomic fractions, with Ni present at slightly favored atomic fractions. For example, Ni may be present in an amount about 1 atomic percent, about 2 atomic percent, about 3 atomic percent, about 4 atomic percent, about 5 atomic percent, or about 6 atomic percent higher than an amount of Ti. In an embodiment, the Ni content is about 2 atomic percent to about 5 atomic percent higher than the amount of Ti.

As described above, at Nb concentrations greater than the Nb solubility limit in Ni—Ti, a dual phase microstructure develops. A preferred dual phase microstructure consists of a Ni—Ti rich first phase with Nb in solution at its solubility limit, and a second phase, consisting of niobium present up to the Ni—Ti—Nb quasi-binary eutectic concentration. Where the appropriate atomic fractions of the bulk alloy are selected, such a dual phase microstructure may naturally arise by virtue of a eutectic reaction during solidification. In other words, because the eutectic composition has the lowest melting temperature, the Ni—Ti rich first phase solidifies out of the melt first, followed by the eutectic mixture of two phases last. Where Nb is present over the quasi-binary eutectic concentration, primary dendrites of the Nb-rich phase solidfy out before the eutectic mixture of two phases. Because the primary Nb-rich dendrites would be typically much coarser than the Nb-rich phase particles that form as part of the lamellar eutectic mixture which solidifies subsequently, its presence may be avoided (by formulating the composition to include no more niobium than that included in the eutectic) or its effect very limited (by ensuring that the Nb concentration is not significantly elevated over the niobium content of the eutectic—e.g., by limiting Nb content to not greater than 30 or 35 atomic percent. Further, the niobium-rich second phase (e.g., the eutectic) is known to be ductile and is coherent with the primary Ni—Ti phase.

The dual phase microstructure may exhibit qualities of a so-called metal matrix composite. The term metal matrix composite (MMC) encompasses a wide range of scales and microstructures; however, the bulk properties of an MMC are typically accounted for by the so-called rule of mixtures, which describes the properties of a composite in terms of a volume weighted average of the properties of each of the individual phases (i.e., the primary and dispersed phases). While the rule of mixtures is to some extent an approximation, it does provide a useful metric for understanding the properties of the dual phase Ni—Ti—Nb alloy systems.

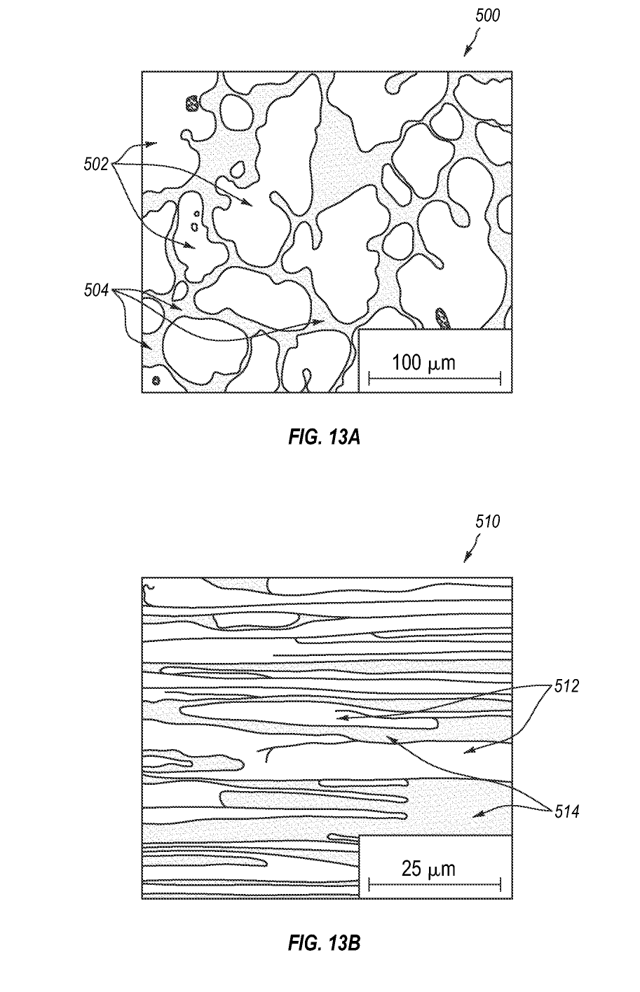

The dual phase Ni—Ti—Nb alloy system includes two ductile phases having widely different mechanical properties. Cast ingots of the Ni—Ti—Nb alloy may contain clusters or primary dendrites of the Ni—Ti rich first phase 502 surrounded by a matrix of a eutectic mixture 504 (FIG. 13A) of the Ni—Ti first phase and the relatively Nb-rich second phase. Upon working down the cast material 500 to produce a guide wire or other intra-corporal body structure (e.g., by one or more of drawing, stamping, rolling, flattening, swaging, or other suitable working techniques), the primary dendrites or clusters 502 begin to be elongate and the eutectic structure becomes elongated while the Nb-rich second phase may become fragmented, resulting in a much more homogeneous microstructure, but which is not completely homogenous, but may include small Nb richer eutectic phase particles surrounded by the Ni—Ti rich first phase. In one embodiment (FIG. 13B), the structure 510 is layered, including thin elongate bands 512 of the primary phase interspersed with thin bands of the eutectic mixture of both phases 514. Such a structure may be directional (e.g., in the direction of the rolling, drawing, etc.).

Where the Nb-rich phase is present, this nearly pure Nb phase (body centered cubic or “bcc” Nb) is coherent with the Ni—Ti phase and both phases are sufficiently ductile to withstand conventional metalworking processes following melting and casting by methods used for commercial binary nitinol. For further discussion of Ni—Ti—Nb alloy systems see, e.g., Eutectic Liquid Formation in the NiTi—Nb System New Joining Method for Nitinol Point, Ke-Bin Low et al., Proceedings of the International Conference on Shape Memory and Superelastic Technologies (2008) pp. 829-836.

The relationship between Nb and Ti contents which serves to maintain the transformation temperature (Mₛ) within a reasonably consistent range is believed to be essentially linear over the range from approximately 15 atomic percent Nb to about 35 atomic percent Nb. This relationship may not be 1:1; but perhaps approximately 0.45:1, which means that Nb may not substitute 1:1 for Ti in the NiTi matrix. Rather, it appears to partition almost equally (a 0.5:1 relationship representing exactly equal substitution for Ti and for Ni). That is, as more and more Nb is added over the range of interest, the necessary reduction in Ti required to maintain a desirable Mₛ transformation temperature is nearly half of the Nb addition (i.e., about 0.45), and the reduction in Ni is also nearly half the Nb addition. As such, it is believed that incremental additions of Nb may generate more of a Nb-richer second phase rather than altering the composition of the NiTi matrix.

In any case, a portion of the Ni, Ti, or perhaps both can be substituted with Nb. In attempting to formulate new Ni—Ti—Nb alloys, a first approximation may be to constrain the Ni and Ti compositions such that the amount of Ni always exceeds the amount of Ti by the amounts described above (e.g., about 2 to about 4 or 5 atomic percentage points higher Ni than Ti). Suitable examples of single or dual phase Ni—Ti—Nb alloys may include about 36.5 atomic percent to about 51 atomic percent Ni, 38 atomic percent to about 51 atomic percent Ni, 44 atomic percent to about 51 atomic percent Ni, 45 atomic percent to about 50 atomic percent Ni, 46 atomic percent to about 49 atomic percent Ni, or from about 47 atomic percent to about 48 atomic percent Ni. In an embodiment, the nickel content may be below 50 atomic percent.

Suitable examples of single or dual phase Ni—Ti—Nb alloys may include about 33.5 atomic percent to about 48 atomic percent Ti, about 36 atomic percent to about 46 atomic percent Ti, about 41 atomic percent to about 48 atomic percent Ti, from about 42 atomic percent to about 47 atomic percent Ti, from about 43 atomic percent to about 46 atomic percent Ti, or from about 44 atomic percent to about 45 atomic percent Ti. In an embodiment, the titanium content may be below 50 atomic percent. The Ni content may be somewhat higher than the Ti content, as described above. Specific examples within the ranges recited herein are shown in Table 1, below.

TABLE 1
Exemplary Ni—Ti—Nb Alloys

| Example | Ti (at %) | Ni (at %) | Nb (at %) | Mₛ (° C.) | Ti/Ni Ratio |
|---|---|---|---|---|---|
| 1 | 48 | 49 | 3 | 29 | 0.98 |
| 2 | 41.5 | 43.5 | 15 | −78 | 0.95 |
| 3 | 40 | 42 | 18 | −92 | 0.95 |
| 4 | 38 | 40 | 22 | −105 | 0.95 |
| 5 | 37 | 39 | 24 | −110 | 0.95 |
| 6 | 36 | 38 | 26 | −120 | 0.95 |
| 7 | 46 | 51 | 3 | <−196 | 0.90 |
| 8 | 47 | 50 | 3 | <29 | 0.94 |
| 9 | 46.5 | 50 | 3.5 | <29 | 0.93 |
| 10 | 37 | 37 | 26 | −11 | 1.0 |
| 11 | 41 | 41 | 18 | −13 | 1.0 |
| 12 | 40 | 40 | 20 | −16 | 1.0 |
| 13 | 39 | 39 | 22 | −16 | 1.0 |
| 14 | 36 | 36 | 28 | −23 | 1.0 |
| 15 | 38 | 38 | 24 | −25 | 1.0 |
| 16 | 35 | 35 | 30 | −38 | 1.0 |
| 17 | 40.5 | 43.5 | 16 | <−196 | 0.93 |
| 18 | 39 | 43 | 18 | <−196 | 0.91 |
| 19 | 39 | 44 | 17 | <−196 | 0.89 |
| 20 | 37 | 41 | 22 | <−196 | 0.90 |
| 21 | 47 | 50 | 3 | N/A | 0.94 |
| 22 | 39.5 | 42.5 | 18 | N/A | 0.93 |
| 23 | 35 | 38 | 27 | N/A | 0.92 |
| 24 | 33.5 | 36.5 | 30 | N/A | 0.92 |

Manipulation of the Ni—Ti ratio may further decrease the martensitic transformation temperature (Mₛ), effectively increasing the plateau stress level due to the increased difference between the Mₛ temperature and the intended service temperature (e.g., body temperature). In an embodiment, preferably, the ratio of Ti to Ni is between 0.88 and about 1, from 0.9 to 0.98, or from 0.92 to 0.96.

In an embodiment, the Mₛ temperature of the Ni—Ti—Nb allow is less than about −5° C., less than about −10° C., less than −15° C., less than −25° C., or less than −40° C. Higher Ni to Ti ratios generally correspond to decreased Mₛ temperature.

Embodiments of the present invention provide guide wire devices and other intra-corporal medical devices that include Ni—Ti alloys which possess substantially greater Young's modulus, shear modulus, and plateau stress levels than comparable binary nitinol.

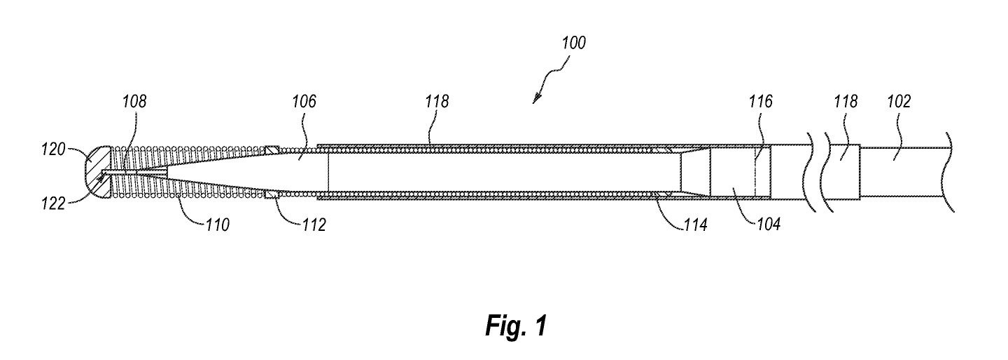

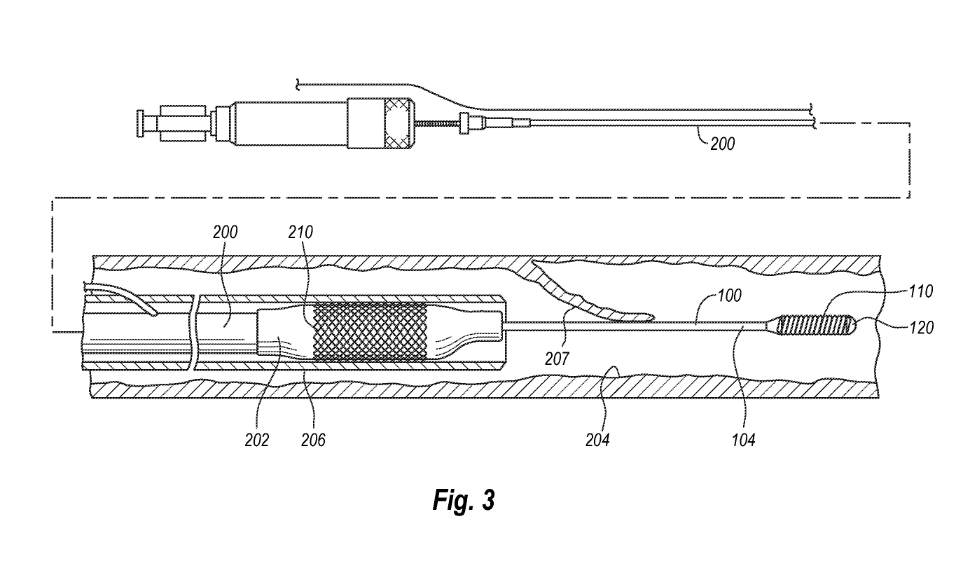

Whether a “low” or “high” Nb Ni—Ti—Nb alloy is employed, the alloy may be cold worked to stabilize the martensitic structure of the Ni—Ti rich first phase. Cold working the Ni—Ti—Nb alloy stabilizes the alloy's martensitic phase and yields a linear pseudo-elastic microstructure where reversion to the austenite phase is retarded or altogether blocked. Where cold working is sufficiently high so as to block the phase change, the Ni—Ti—Nb alloy exhibits linear pseudo-elastic behavior with increased elastic modulus (e.g., where elastic moduli values are increased even further). Because the phase change is blocked, less applied strain is required to attain permanent deformation so that guide wires made from material in this condition are more readily shapeable by the end user (e.g., a practitioner may form a J-bend in a distal tip of a linear pseudo-elastic Ni—Ti—Nb alloy guide wire as will be described below in conjunction with FIGS. 1 and 3). Alloys having linear pseudo-elastic characteristics and a high elastic modulus and shear modulus facilitate excellent torque transmission, steerability, shapeability, stent scaffolding strength, and similar characteristics associated with high stiffness that are desirable in various intra-corporal medical devices. While providing excellent stiffness characteristics, the linear pseudo-elastic Ni—Ti—Nb alloys also exhibit extensive, recoverable strain, which greatly minimizes the risk of performance loss due to kinking with possible concomitant damage to body lumens during the advancement of a guide wire or other device therein.

### IV. Intra-Corporal Medical Devices

In ordinary applications, differences in elastic modulus between two materials can be readily compensated for by dimensional alterations. That is, for example, the inherent floppiness of a wire material that has a low elastic modulus can ordinarily be compensated for by increasing the diameter of the wire in order to attain equivalent deflection behavior when compared to a wire material with a higher elastic modulus. However, guide wire devices, stents, embolic protection filters, graft assemblies, and similar intra-corporal medical devices typically face inherent dimensional constraints that are imposed by the overall product profile, the size of the anatomy to be accessed and similar factors. For this reason, the Ni—Ti—Nb alloys discussed herein, which have higher stiffness characteristics than comparable binary Ni—Ti, significantly expand the maximum range of torsional or bending stiffness that can be achieved in a guide wire or other intra-corporal medical device of a given profile.

Intra-corporal medical devices include, but are not limited to, guide wires, stents, embolic protection filters, and graft assemblies. Such devices (or portions thereof) can be formed from the described Ni—Ti—Nb alloys so as to benefit from increased Young's modulus, shear modulus, and plateau stress levels. For example, guide wire devices are used in minimal invasive procedures such as, but not limited to, percutaneous transluminal coronary angioplasty (PTCA) to track through vessels, access and cross lesions, and support interventional devices for a variety of procedures. Guide wire devices have a number of desired performance characteristics such as, but not limited to, flexibility, support, the ability to steer the guide wire device through the patient's vasculature (i.e., trackability), the ability to transmit steering torque from the proximal end of the device outside the patient's body to the distal tip inside (i.e., torqueability), torque control, lubricity, the ability to visualize the guide wire device as it progresses through the patient's body, and tactile feedback. Guide wire design typically involves the balancing of these various characteristics.

In order to, for example, track through a patient's vasculature, guide wire devices are quite long and thin. In terms of length, guide wire devices need to be long enough to travel from an access point outside a patient's body to a treatment site and narrow enough to pass freely through the patient's vasculature. Lengths of about 150 cm to about 300 cm are typical. In terms of diameter, typical guide wire devices have an overall diameter of about 0.2 mm to about 0.5 mm for coronary use. Larger diameter guide wires may be employed in peripheral arteries and other relatively larger body lumens. The diameter of the guide wire device affects its flexibility, support, and torque. Thinner wires are more flexible and are able to access narrower vessels while larger diameter wires offer greater support and torque transmission. While stiffness, elastic modulus, and shear modulus may be increased by increasing wire diameter, such larger diameter wires are not physically sized to be compatible with associated devices such as balloon dilation catheters and stent delivery systems, or may not be readily insertable into the partially occluded vasculature of some patients. As such, materials properties, rather than physical size, can be manipulated in order to achieve more desirable stiffness characteristics.

Requirements for stents, embolic protection filters, graft assemblies and similar intra-corporal medical devices similarly benefit from increased Young's modulus, shear modulus (together herein referred to as elastic moduli), as well as a higher plateau stress level in cases where the alloy is processed to attain super-elastic characteristics.

A. Guide Wire Devices

Referring now to FIG. 1, a partial cut-away view of an example of a guide wire device 100 that embodies features of the invention is illustrated. The guide wire device 100 may be adapted to be inserted into a patient's body lumen, such as an artery or another blood vessel. The guide wire device 100 includes an elongated proximal portion 102 and a distal portion 104. In one embodiment, both the elongated proximal portion 102 and the distal portion 104 may be formed from a Ni—Ti—Nb alloy. In another embodiment, the elongated proximal portion 102 may be formed from a first material such as stainless steel (e.g., 316L stainless steel) or a Ni—Ti alloy and the distal portion may be formed from a second material such as a Ni—Ti—Nb alloy. In embodiments where the elongated proximal portion 102 and the distal portion 104 are formed from different materials, the elongated proximal portion 102 and the distal portion 104 may coupled to one another via a welded or other joint 116 that couples the proximal portion 102 and the distal portion 104 into a torque transmitting relationship.

In an embodiment, selected portions of the guide wire device 100 or the entire guide wire device 100 may be cold worked in order to yield a linear pseudo-elastic microstructure. As mentioned, increasing levels of cold-work (i.e., permanent deformation without subsequent heat treatment) progressively raises the yield strength of the material and leads to almost the complete disappearance of austenite and the elimination of the plateau (austenite to martensite transformation) on the stress strain curve, resulting in a unique stress strain curve without a classic linear modulus of elasticity and without an apparent yield point. In other embodiments, a lower level of cold work, no cold work, and/or subsequent heat treatments may be provided, maintaining the super-elastic characteristics of the Ni—Ti—Nb alloy.

For example, in an embodiment, selected portions of the guide wire device 100 or the entire guide wire device 100 may be cold worked to impart a linear pseudo-elastic microstructure that includes at least about 40% cold work, or at least about 50% cold work. In another example, about 20% to about 90% cold work, about 30% to about 65% cold work, about 40% cold work to about 50% cold work, or about 45% cold work may be provided. Depending on the composition of the Ni—Ti—Nb alloy and the amount of cold work, the Ni—Ti—Nb alloy may have an elastic modulus of about 50 gigapascals (GPa) to about 100 GPa or an elastic modulus of about 60 GPa to about 70 GPa. In an embodiment, the Ni—Ti—Nb alloy exhibits a Young's modulus in an austenite phase that is greater than about 85 GPa (greater than binary Ni—Ti), and a Young's modulus in a martensite phase that is greater than about 42 GPa (greater than binary Ni—Ti).

Referring again to FIG. 1, the distal portion 104 may have at least one tapered section 106 that, in the illustrated embodiment, becomes smaller in the distal direction. The length and diameter of the tapered distal core section 106 can, for example, affect the trackability of the guide wire device 100. Typically, gradual or long tapers produce a guide wire device with less support but greater trackability, while abrupt or short tapers produce a guide wire device that provides greater support but also greater risk of prolapse (i.e., kink) when steering.

In the illustrated embodiment, the tapered distal core section 106 may further include a shapeable distal end section 108. Ni—Ti alloys such as Ni—Ti—Nb are shapeable in the linear pseudo-elastic state. The linear pseudo-elastic state can be imparted to the Ni—Ti alloy by cold work, with varying amounts of cold work imparting different degrees of linear pseudo-elasticity and differing degrees of shapeability. In contrast to superelastic Ni—Ti alloy, linear pseudo-elastic Ni—Ti alloy can readily be permanently deformed by deforming the material beyond its elastic strain limit. As such, the shapeable distal end section 108 can allow a practitioner to shape the distal and of the guide wire device 100 to a desired shape (e.g., a J-bend) for tracking through the patient's vasculature.

In an embodiment, the shapeable distal end section 108 is manufactured by grinding the distal end of the Ni—Ti distal section 104 to a first cross-sectional dimension (e.g., by centerless grinding) and cold-working (e.g., by flattening) the ground portion to a second cross-sectional dimension. For example, the first dimension can be in a range from about 0.1 mm to about 0.07 mm, or about 0.08 mm. The second cross-sectional dimension, which is cold worked by, for example, flattening at least a part of the ground distal section, may be in a range from about 0.065 mm to about 0.008 mm, about 0.055 mm to about 0.03 mm, about 0.05 to about 0.04 mm, or about 0.045 mm.

The length of the distal end section 106 can, for example, affect the steerability of the guide-wire device 100. In one embodiment, the distal end section 106 is about 10 cm to about 40 cm in length. In another embodiment, the distal end section 106 is about 2 to about 6 cm in length, or about 2 to 4 cm in length.

As illustrated in FIG. 1, the guide wire device 100 includes a helical coil section 110. The helical coil section 110 affects support, trackability, and visibility of the guide wire device and provides tactile feedback. In some embodiments, the most distal section of the helical coil section 110 is made of radiopaque metal, such as platinum or a platinum-nickel or platinum-iridium alloy, to facilitate the observation thereof while it is disposed within a patient's body. As illustrated, the helical coil section 110 may be disposed about at least a portion of the distal portion 104 and may have a rounded, atraumatic cap section 120 on the distal end thereof. The helical coil section 110 may be secured to the distal portion 104 at proximal location 114 and at intermediate location 112 by a suitable technique such as, but not limited to, soldering, brazing, or welding.

In one embodiment, the distal end section 108 may be secured to the rounded, atraumatic cap section 120 by virtue of a joint 122 such as, but not limited to, a soldered, brazed, or welded joint. Because Ni—Ti alloy forms a persistent oxide layer, it can be difficult to solder Ni—Ti. Therefore, in one embodiment, the distal end section 108 may be joined to the atraumatic cap section 120 using a soldering technique specially adapted to soldering Ni—Ti alloys. Briefly stated here, the distal end section 108 may be prepared and a layer of solder material may be applied thereto and the distal end section 108 may be soldered to the rounded, atraumatic cap section 120 to form a soldered joint 122.

In one embodiment, portions of the guide wire device 100 are coated with a coating 118 of lubricous material such as polytetrafluoroethylene (PTFE) (sold under the trademark Teflon by du Pont, de Nemours & Co.) or other suitable lubricous coatings such as the polysiloxane coatings, polyvinylpyrrolidone (PVP), and the like.

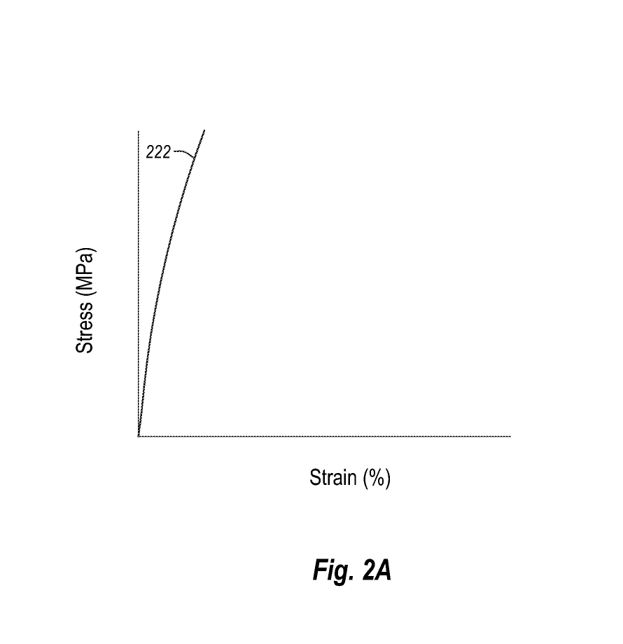

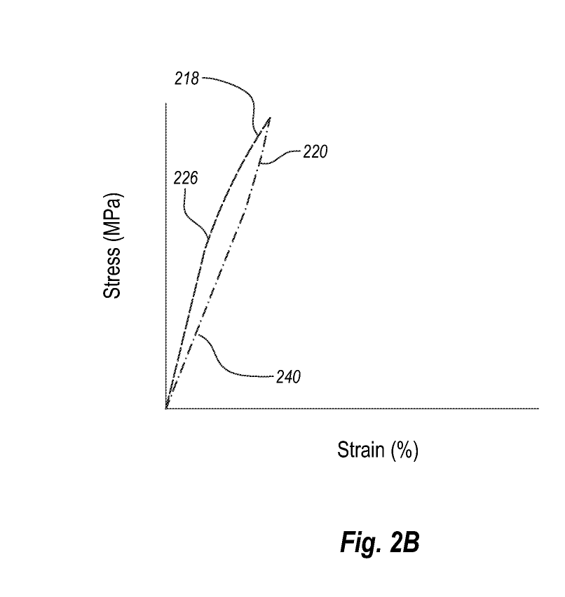

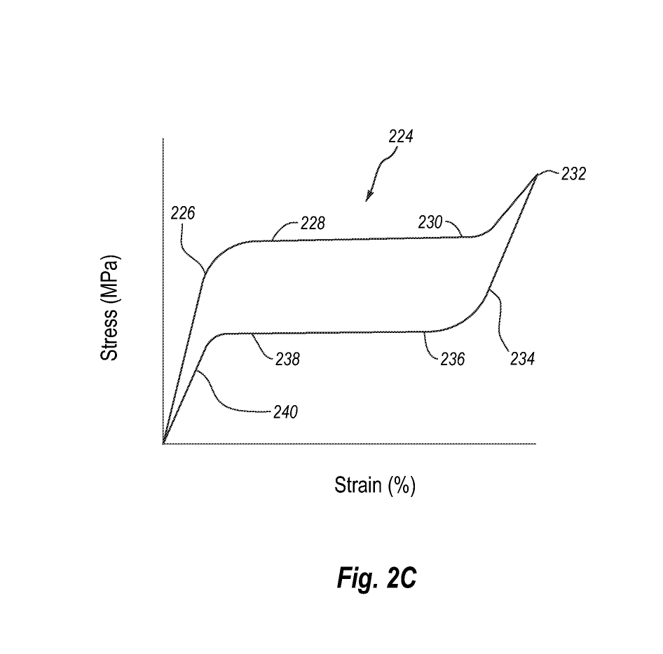

To illustrate the foregoing points, FIGS. 2A-2C show the elastic component of three idealized stress-strain curves for 316L stainless steel (FIG. 2A—curve 222), a linear pseudo-elastic Ni—Ti—Nb alloy (FIG. 2B—curves 218 and 220), and a super-elastic Ni—Ti alloy (FIG. 2C—curve 224). The stress/strain relationship is plotted on x-y axes, with the x axis representing strain and the y axis representing stress.

In curve 224, when stress is applied to a specimen of a metal such as Ni—Ti or a Ni—Ti alloy exhibiting super-elastic characteristics at a temperature at or above the temperature at which the transformation of the martensitic phase to the austenitic phase is complete, the specimen deforms elastically (curve portion 226) until it reaches a particular stress level where the alloy then undergoes a stress-induced phase transformation from the austenitic phase to the martensitic phase (i.e., the stress-induced martensite phase). As the phase transformation progresses, the alloy undergoes significant increases in strain with little or no corresponding increases in stress. On curve 224, this is represented by the upper, nearly flat stress plateau 228 (e.g., at approximately 70 to 80 ksi). The strain increases while the stress remains essentially constant until the transformation of the austenitic phase to the martensitic phase is complete (at curve portion 230). Thereafter, further increase in stress is necessary to cause further deformation (curve portion 232). The martensitic metal first yields elastically upon the application of additional stress and then plastically with permanent residual deformation (not shown).

If the load on the specimen is removed before any permanent deformation has occurred, the martensite specimen elastically recovers and transforms back to the austenitic phase. The reduction in stress first causes a decrease in stress (curve portion 234). As stress reduction reaches the level at which the martensitic phase transforms back into the austenitic phase (curve portion 236), the stress level in the specimen remains essentially constant (curve portion 238), but at a lower level than the constant stress level at which the reverse transformation occurred. In other words, there is significant recovery in strain with only negligible corresponding stress reduction. This is represented in curve 224 by the lower stress plateau 238 (e.g., at about 20 ksi).

After the transformation back to austenite is complete, further stress reduction results in elastic strain reduction (curve portion 240). This ability to incur significant strain at relatively constant stress upon the application of a load and to recover from the deformation upon the removal of the load is commonly referred to as super-elasticity or non-linear pseudo-elasticity. The area between or bounded by the upper plateau 228 and lower plateau 238 represents the hysteresis in the super-elastic Ni—Ti alloy.

FIG. 2B shows a curve 218-220 representing the idealized behavior of Ni—Ti—Nb alloy which has been cold worked so as to inhibit any further stress induced phase transformation (i.e., it exhibits so called linear pseudo-elastic behavior). While curves 218 and 220 may be typically be described as “linear” by those in the art, it is readily apparent that the name is somewhat of a misnomer, as there may be noticeable curvature to the curve. Curve 218-220 does not contain any flat plateau stresses, as found in super-elastic curve 224. This stands to reason since the Ni—Ti—Nb alloy of curve 218-220 remains in the martensitic phase throughout stress loading and unloading, and does not undergo any phase change. Curve 218-220 shows that increasing stress begets a proportional increase in reversible strain, and a release of stress begets a proportional decrease in strain. The area bounded between curves 218 and 220 represent the hysteresis in the linear pseudo-elastic Ni—Ti alloy.

With the use of a linear pseudo-elastic Ni—Ti—Nb alloy, the mechanical strength of the disclosed medical devices may be substantially greater per unit strain than a comparable device made of super-elastic Ni—Ti alloy. Consequently, a major benefit may be that smaller component parts (e.g., such as the shapeable distal end section 108) can be used. A small profile can be a very important factor for crossing narrow lesions or for accessing remote and tortuous arteries.

Even where the Ni—Ti—Nb alloy retains super-elastic properties, because of the inclusion of the Nb, the elastic moduli values for the Ni—Ti—Nb are significantly increased as compared to comparable super-elastic binary Ni—Ti. The increased stiffness provides the desired increased torsional transmitting ability, increased stent scaffolding strength, etc., all without requiring increased physical dimensions.

FIG. 2A shows curve 222 which represents the conventional elastic behavior of a standard 316L stainless steel. Stress is incrementally applied to the steel and, just prior to the metal deforming plastically, incrementally released.

Referring now to FIG. 3, the guide wire device 100 is shown configured to facilitate deploying a stent 210. FIG. 3 provides more detail about the manner in which the guide wire device 100 may be used to track through a patient's vasculature where it can be used to facilitate deployment of a treatment device such as, but not limited to, stent 210. FIG. 3 illustrates a side elevation view, in partial cross-section, of a delivery catheter 200 having a stent 210 disposed thereabout according to an embodiment of the present disclosure. The portion of the illustrated guide wire device 100 that can be seen in FIG. 3 includes the distal portion 104, the helical coil section 110, and the atraumatic cap section 120. The delivery catheter 200 may have an expandable member or balloon 202 for expanding the stent 210, on which the stent 210 is mounted, within a body lumen 204 such as an artery. In another embodiment, stent 210 may be self-expanding. For example, a sheath may be initially disposed over stent 210 so as to maintain an un-expanded configuration. When stent 210 is advanced to a desired position, the sheath may be removed and stent 210 expanded.

The delivery catheter 200 may be a conventional balloon dilatation catheter commonly used for angioplasty procedures. The balloon 202 may be formed of, for example, polyethylene, polyethylene terephthalate, polyvinylchloride, nylon, Pebax™ or another suitable polymeric material. To facilitate the stent 210 remaining in place on the balloon 202 during delivery to the site of the damage within the body lumen 204, the stent 210 may be compressed onto the balloon 202. Other techniques for securing the stent 210 onto the balloon 202 may also be used, such as providing collars or ridges on edges of a working portion (i.e., a cylindrical portion) of the balloon 202.

In use, the stent 210 may be mounted onto the inflatable balloon 202 on the distal extremity of the delivery catheter 200. The balloon 202 may be slightly inflated to secure the stent 210 onto an exterior of the balloon 202. The catheter/stent assembly may be introduced within a living subject using a conventional Seldinger technique through a guiding catheter 206. The guide wire 100 may be disposed across the damaged arterial section with the detached or dissected lining 207 and then the catheter/stent assembly may be advanced over the guide wire 100 within the body lumen 204 until the stent 210 is directly under the detached lining 207. The balloon 202 of the catheter 200 may be expanded, expanding the stent 210 against the interior surface defining the body lumen 204 by, for example, permanent plastic deformation of the stent 210. In an embodiment employing a self-expanding stent, removal of a sheath may be sufficient to allow a self-expanding stent to expand against the interior surface defining body lumen 204. In either case, when deployed, the stent 210 holds open the body lumen 204 after the catheter 200 and the balloon 202 are withdrawn.

B. Stent Devices

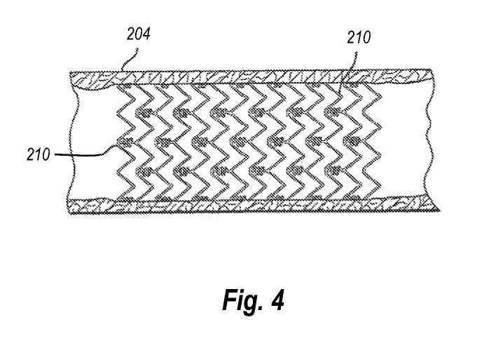

As depicted in FIG. 4, the implanted stent 210 remains in the vessel 204 after the balloon 202 has been deflated and the catheter 200 and guide wire 100 have been withdrawn from the patient.

The stent 210 (which may be formed of the disclosed Ni—Ti—Nb alloys) serves to hold open the body lumen 204 after the catheter 200 is withdrawn. Such a stent 210 may be fabricated from an elongated tubular member, where the undulating components of the stent are relatively flat in transverse cross section, so that when the stent 210 is expanded, it is pressed into the wall of the body lumen and as a result does not interfere with the blood flow through the body lumen 204. The stent 210 may be pressed into the wall of the body lumen and may eventually be covered with endothelial cell growth, which further minimizes blood flow interference. The undulating ring portion of the stent 210 provides good tacking characteristics to prevent stent movement within the body lumen. Stent 210 may include closely spaced cylindrical elements at regular intervals for providing uniform support for the wall of the body lumen. Such a configuration may better serve to tack up and hold in place small flaps or dissections in the wall of the body lumen, as illustrated in FIG. 4.

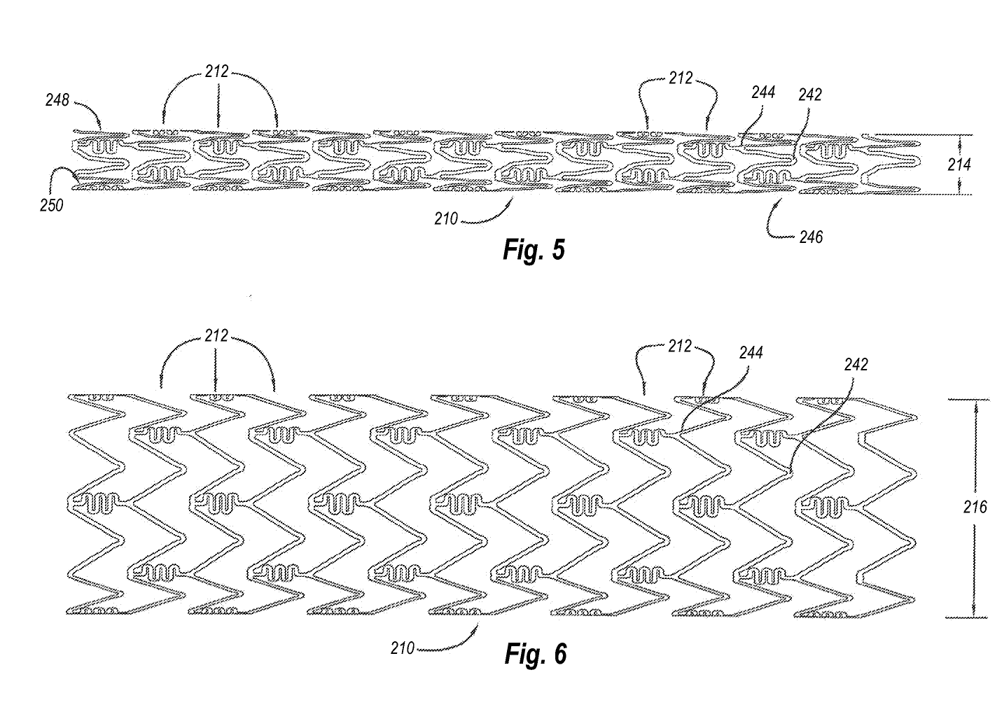

As shown in FIGS. 5-9, the stent 210 may be made up of a plurality of cylindrical rings 212, which extend circumferentially around the stent. The stent has a delivery diameter 214 (FIG. 5), and an implanted diameter 216 (FIG. 6). Each cylindrical ring 212 has a proximal end 242 and a distal end 244. Where the stent is laser cut from a solid tube, there may be no discreet parts, such as the described cylindrical rings. However, it may be beneficial for identification and reference to various parts to refer to the cylindrical rings and the following parts of the stent.

Each cylindrical ring 212 defines a cylindrical plane 246, which is bound by the cylindrical ring proximal end 242, the cylindrical ring distal end 244 and the circumferential extent as the cylindrical ring 212 traverses around the cylinder. Each cylindrical ring includes a cylindrical outer wall surface 248, which defines the outer most surface of the stent 210, and a cylindrical inner wall surface 250, which may define the innermost surface of the stent. The cylindrical plane may follow the cylindrical outer wall surface.

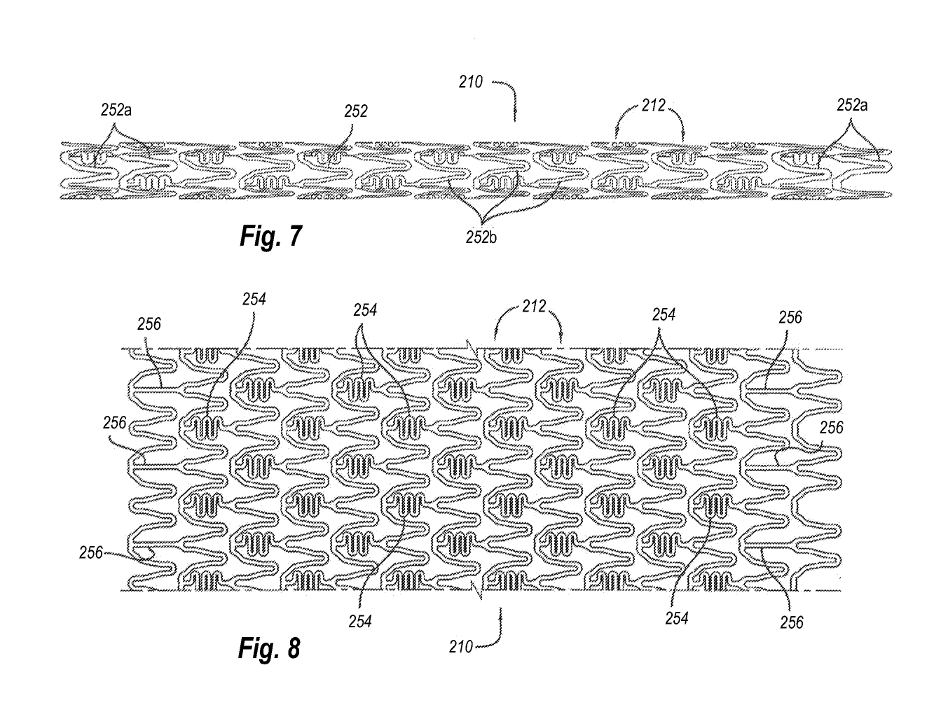

As shown in FIGS. 7 and 8, the stent 210 may be constructed with struts 252 formed from Ni—Ti—Nb alloy. In an example, struts 252a at the ends of the stent may be thicker than the struts 252b in the center of the stent 210 for purposes for increased radiopacity and to counter non-uniform balloon expansion. When the balloon first inflates, the balloon ends have a tendency to inflate at a faster rate than the balloon center. However, with thicker struts at the stent ends, the balloon, and hence the stent, will expand more uniformly. In an embodiment, stent 210 may comprise a linear pseudo-elastic Ni—Ti—Nb alloy.

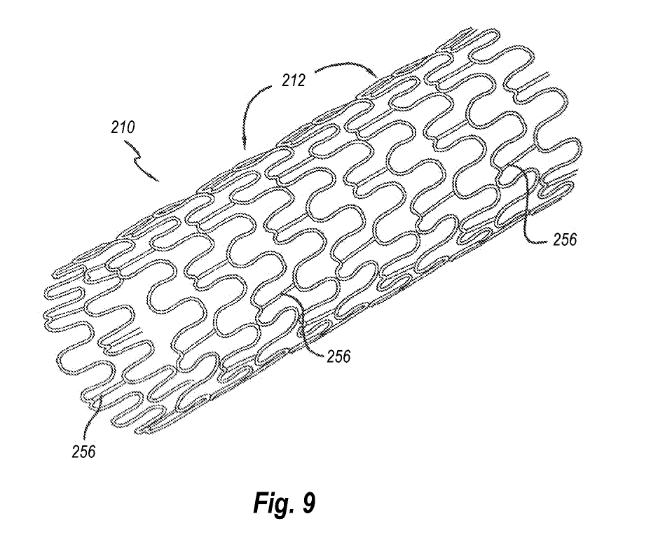

Referring to FIGS. 6, 8 and 9, each adjacent cylindrical ring 212 may be connected by at least one undulating link 254 or straight link 256. In an embodiment, the stent may include only straight links (FIG. 9), may include only undulating links (FIG. 6) or may include both undulating links and straight links (FIG. 8) to connect adjacent cylindrical rings. Both the straight links and the undulating links assist in preventing stent foreshortening. Further, the straight links may provide more stability and rigidity in a localized area, such as at the stent ends, such that it may be desirable to incorporate more straight links between the cylindrical rings at the stent ends, than in the center of the stent. An undulating link may be positioned substantially within the cylindrical plane 246, as defined by the cylindrical outer wall surface 248 and the cylindrical inner wall surface 250.

The stent 210 can be made in many ways. One method of making the stent is to cut a thin-walled tube of material to remove portions of the tubing in the desired pattern for the stent, leaving relatively untouched the portions of the metallic tubing that are to form the stent. Cutting of the tubing in the desired pattern may be by means of a machine-controlled laser. Other methods of forming the stent can be used, such as chemical etching; electric discharge machining; laser cutting a flat sheet and rolling it into a cylinder with a longitudinal weld; and the like. In addition, the stent and/or its struts may be formed from a wire or elongated fiber constructed from a Ni—Ti—Nb material. The cross-section of such struts may be round, rectangular or any other suitable shape for constructing a stent.

C. Embolic Protection Devices

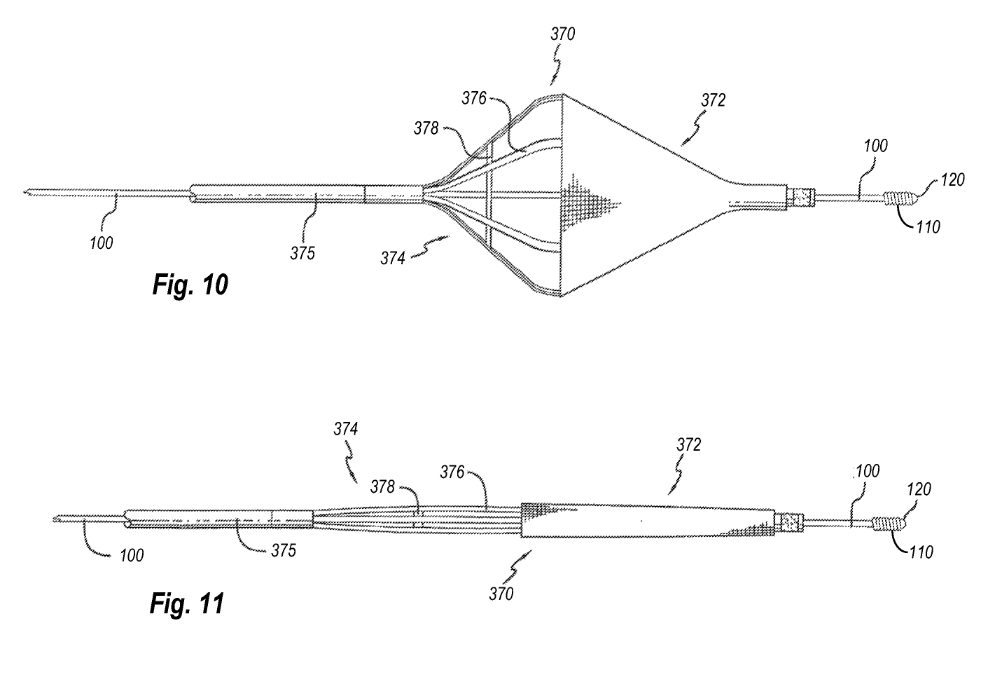

Referring now to FIGS. 10 and 11, and by way of example, the Ni—Ti—Nb alloys described herein may be employed in fabrication of an embolic protection device 370. Such device may include a filter assembly 372 and expandable strut assembly 374. The embolic protection device may further include an elongated tubular member 375, within which may be disposed a guide wire 100 for positioning the device within a body lumen. The embolic protection device may include a plurality of longitudinal struts 376 and transverse struts 378 that may be fabricated at least in part from Ni—Ti—Nb alloys according to the present disclosure. In addition, other components of the filter assembly may be formed from a Ni—Ti—Nb alloy as heretofore described. As described above, guide wire 100 (including distal end 110 and/or 120) may include or be constructed from a Ni—Ti—Nb alloy.

D. Graft Devices

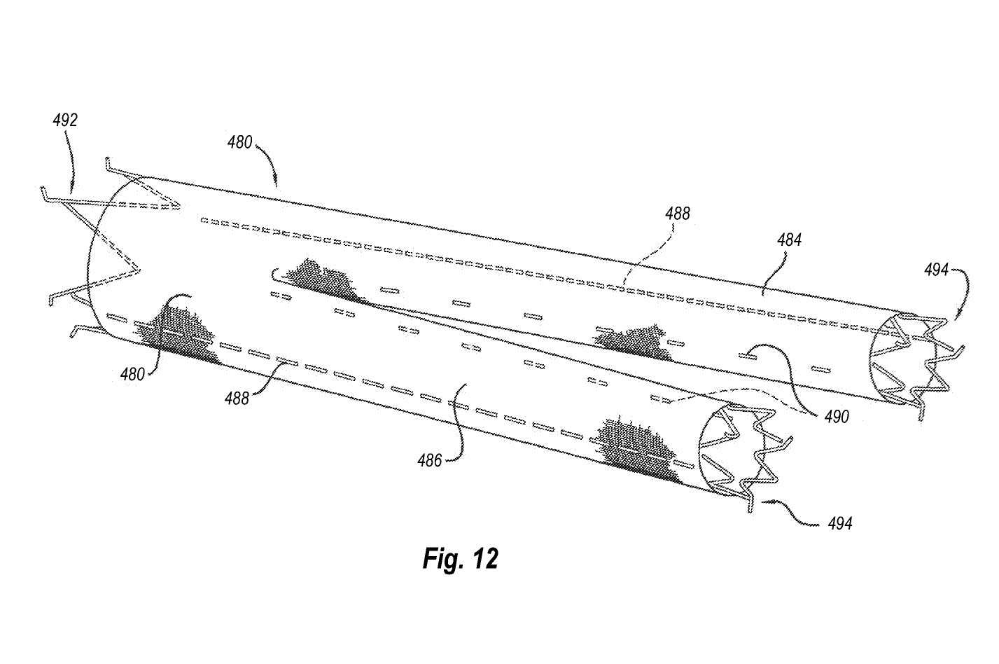

Referring now to FIG. 12, Ni—Ti—Nb alloys as described herein may be incorporated into a bifurcated graft 480 or a tubular graft (not shown). Such a graft may include a DACRON, TEFLON or other suitable flexible material having an upper body 482, a first leg 484 and a second leg 486, wherein the legs are joined to the upper body. Such a configuration forms a “Y” or “pants leg” configuration. A plurality of closely spaced markers 488 formed from a radiopaque material (e.g., which may be Ni—Ti—Nb) may be provided on the outside of the first and second legs. Similarly, wider spaced markers 490 may be provided on the inside of the legs of the bifurcated graft (or vice versa). Such markers may be formed from Ni—Ti—Nb or other radiopaque materials, which may be sewn, glued or otherwise bonded to the graft.

In many such grafts 480, such as those used for repairing an abdominal aortic aneurysm, the upper body may include a first attachment system 492 positioned proximate to an upper opening of the graft. Tube grafts may contain a like attachment system at the lower opening of the graft. Similarly, bifurcated grafts may include smaller attachment systems 494 positioned at the end of the legs and proximate to the lower openings of the graft. As heretofore described regarding other intra-corporal medical devices, the attachment systems may be made of Ni—Ti—Nb alloy in accordance with the present disclosure. Such stents and attachment systems may be of various configurations, such as, but not limited to, a ring and link design, a zigzag design, a coil design or a tubular mesh design.

While particular intra-corporal medical devices that may benefit from fabrication from Ni—Ti—Nb alloys have been illustrated and described, it will be apparent to those skilled in the art that other intra-corporal medical devices may be formed from such alloys. Likewise, the invention is not limited to any particular method of forming the under lying medical device structure.

### V. Methods for Fabricating Intra-Corporal Medical Devices

In an embodiment, a method for fabricating an intra-corporal medical device such as a guide wire device is disclosed. The method includes (1) fabricating a body of the device (e.g., an elongated shaft member that includes a proximal section and a distal section in the case of a guide wire). In one embodiment, at least a portion of the body includes a nickel-titanium (Ni—Ti) alloy that includes nickel (Ni), titanium (Ti), and niobium (Nb). The method may further include (2) cold working at least the Ni—Ti alloy. In an embodiment, sufficient cold working may be done so that the body exhibits linear pseudo-elastic behavior in the martensitic phase.

In another embodiment, a method for fabricating an intra-corporal device includes fabricating a device body, wherein at least a portion of the body comprises a Ni—Ti—Nb alloy comprising Ni, Ti, and Nb. The Nb may be present in the Ni—Ti—Nb alloy in an amount of at least 15 atomic percent so that the Ni—Ti—Nb alloy comprises a first phase that is Ni—Ti rich and a second phase that is richer in Nb than the first phase. The second phase exhibits conventional elastic properties rather than super-elastic properties. Referring to FIG. 13A, prior to cold working, the second phase 504 coexists with the first phase as a eutectic mixture which is surrounded by dendrites of the Ni—Ti rich first phase 502, the Nb richer second phase exhibiting conventional elastic properties while the Ni—Ti rich first phase exhibits super-elastic properties. The dual alloy body 500 is cold worked to yield a structure 510 in which the Ni—Ti rich first phase and the eutectic mixture of both phases are arranged in elongate bands, as seen in FIG. 13B.

In one embodiment, the body can be fabricated from a billet or ingot of the Ni—Ti—Nb alloy using at least one of drawing, rolling, stamping, or other procedures which may result in the desired lamellar structure by which the differing properties of the two phases may be synergized in a thin-layered composite structure where the relatively stiff second phase 514 is present only in small dimensioned particles or thin layers surrounded by the super-elastic or pseudo-elastic Ni—Ti rich first phase 512.

In one embodiment, the cold-worked section(s) may include about 20% to about 90% cold work, about 30% to about 65% cold work, about 40% cold work to about 50% cold work, or about 45% cold work. The cold work imparts even further increased elastic moduli values and further increased plateau stress levels (where super-elastic characteristics are retained). The cold work may be sufficient to impart a martensitic phase having a linear pseudo-elastic microstructure with linear pseudo-elastic behavior without a phase transformation or onset of stress-induced martensite. In one embodiment, the martensitic phase is enhanced and/or stabilized by the cold working.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An intra-corporal medical device comprising:
a body;
at least a portion of the body being fabricated from a nickel-titanium (Ni—Ti) alloy comprising nickel (Ni), titanium (Ti), and niobium (Nb),
wherein the Nb is present in the Ni—Ti alloy in an amount not more than a solubility limit of the Nb in Ni—Ti so that the alloy comprises only a single phase.

2. The medical device of claim 1, wherein Nb is present in the Ni—Ti alloy in an amount of 3.5 atomic percent or less.

3. The medical device of claim 1, wherein the Ni—Ti alloy has a martensite transformation (Mₛ) temperature of less than about −5° C.

4. The medical device of claim 1, wherein Ni is present in the Ni—Ti alloy in an amount that is about 3 atomic percentage points higher than the amount of Ti.

5. The medical device of claim 1, wherein the Ni—Ti alloy exhibits a Young's modulus in both an austenite phase and in a martensite phase that is higher than a Young's modulus of a binary Ni—Ti alloy otherwise similar to the Ni—Ti—Nb alloy but without the Nb.

6. The medical device of claim 5, wherein the Ni—Ti alloy exhibits a Young's modulus in an austenite phase that is greater than about 85 GPa and a Young's modulus in a martensite phase that is greater than about 42 GPa.

7. The medical device of claim 1, wherein the Ni—Ti alloy is a ternary alloy consisting of Ni, Ti, and Nb.

8. An intra-corporal medical device comprising:
a body;
at least a portion of the body being fabricated from a nickel-titanium (Ni—Ti) alloy comprising nickel (Ni), titanium (Ti), and niobium (Nb),
wherein the Nb is present in the Ni—Ti alloy in an amount of at least 15 atomic percent, the Ni—Ti—Nb alloy comprising a primary phase that is Ni—Ti rich and a second phase that is Nb rich, the second phase exhibiting conventional elastic properties rather than super-elastic properties.

9. The medical device of claim 8, wherein the Ni—Ti rich primary phase exhibits super-elastic properties.

10. The medical device of claim 8, wherein the Ni—Ti rich primary phase exhibits linear pseudo-elastic properties as a result of a stress-induced martensitic transformation.

11. The medical device of claim 8, wherein the Nb is present in the Ni—Ti alloy in an amount that is not more than a Nb atomic percentage present in a eutectic Ni—Ti—Nb composition.

12. The medical device of claim 11, wherein Nb is present in the Ni—Ti alloy in an amount of 26 atomic percent or less.

13. The medical device of claim 8, wherein the Ni—Ti alloy has a martensite transformation (Mₛ) temperature of less than about −5° C.

14. The medical device of claim 8, wherein Ni is present in the Ni—Ti alloy in an amount that is about 3 atomic percentage points higher than the amount of Ti.

15. The medical device of claim 8, wherein the Ni—Ti alloy exhibits a Young's modulus in both an austenite phase and in a martensite phase that is higher than a Young's modulus of a binary Ni—Ti alloy otherwise similar to the Ni—Ti—Nb alloy but without the Nb.

16. A method for fabricating an intra-corporal medical device, the method comprising:
fabricating a medical device body, wherein at least a portion of the medical device body comprises a nickel-titanium (Ni—Ti) alloy comprising nickel (Ni), titanium (Ti), and niobium (Nb),
wherein the Nb is present in the Ni—Ti alloy in an amount of at least 15 atomic percent, the Ni—Ti—Nb alloy comprising a primary phase that is Ni—Ti rich and a second phase that is Nb rich, the second phase exhibiting conventional elastic properties rather than super-elastic properties;
prior to hot or cold working, an as-cast microstructure containing the Ni—Ti rich primary phase and a eutectic mixture comprised of both phases; and
cold working the Ni—Ti alloy comprising the primary phase and eutectic mixture to yield a structure in which the Ni—Ti rich primary phase and the eutectic mixture become substantially aligned in the working direction with elongate bands of the primary phase and the eutectic mixture interspersed relative to one another.

17. The method of claim 16, wherein a degree of cold working is sufficient to stabilize the Ni—Ti rich primary phase so that the resulting intra-corporal medical device exhibits linear pseudo-elastic behavior rather than super-elastic behavior.

18. The method of claim 16, wherein a degree of cold working is limited so that the Ni—Ti rich primary phase retains an austenitic structure so that the resulting intra-corporal medical device exhibits super-elastic behavior.

19. The method of claim 16, further comprising heat treating the cold worked Ni—Ti alloy so that the Ni—Ti rich primary phase exhibits an austenitic structure so that the resulting intra-corporal medical device exhibits super-elastic behavior.
